(19) United States
(12) Patent Application Publication
(10) Pub. No.: US 2023/0093073 A1
(43) Pub. Date: Mar. 23, 2023

(54) METHODS AND COMPOSITIONS FOR COMMUNITY-BASED SCREENING OF POLYMICROBIAL INFECTIONS

(71) Applicant: REGENTS OF THE UNIVERSITY OF MINNESOTA, MINNEAPOLIS, MN (US)

(72) Inventors: Ryan HUNTER, Minneapolis, MN (US); Jeffrey FLYNN, Minneapolis, MN (US); Lydia CAMERON, Minneapolis, MN (US)

(73) Assignee: REGENTS OF THE UNIVERSITY OF MINNESOTA, MINNEAPOLIS, MN (US)

(21) Appl. No.: 17/798,666

(22) PCT Filed: Feb. 12, 2021
(86) PCT No.: PCT/US2021/017833

§ 371 Date: Aug. 10, 2022

Related U.S. Application Data
(60) Provisional application No. 62/975,428, filed on Feb. 12, 2020.

(51) Int. Cl. C12Q 1/689; C12Q 1/20; B01L 3/00

(52) U.S. Cl. CPC C12Q 1/689; C12Q 1/20; B01L 3/50853; C12Q 1/6851

(57) ABSTRACT

Provided herein are methods and compositions for community-based screening of polymicrobial infections for the selection and administration of antibiotics and antibiotic combinations for treating infections associated with diseases such as cystic fibrosis, chronic obstructive pulmonary disease, and chronic sinus infections. The methods and compositions of this disclosure are advantageous in the sense that antibiotic effectiveness is assessed with respect to a community of microbes rather than a single microbial population, thus reflecting more accurately the in vivo disease environment and enabling the identification of beneficial antimicrobial therapeutics even when the primary bacterial strain shows antibiotic resistance.

### CROSS REFERENCE TO RELATED APPLICATIONS

This application claims priority to U.S. Provisional Application No. 62/975,428 filed on Feb. 12, 2020, the contents of which are incorporated by reference in its entirety.

### STATEMENT REGARDING FEDERALLY FUNDED RESEARCH

This invention was made with government support under 1R01HL136919 awarded by the National Institutes of Health. The government has certain rights in the invention.

### BACKGROUND

Antimicrobial susceptibility testing (AST) is typically performed under defined conditions delineated by organizations such as the Clinical and Laboratories Standards Institute (CLSI) and the US Food and Drug Administration. In the “gold standard” AST practices, doubling dilutions of antibiotics are mixed with microorganisms, suspended at a particular concentration in a liquid growth medium. The minimal concentration of an antimicrobial that causes visible growth inhibition after a pre-determined incubation period is deemed to be the “minimal inhibitory concentration” (MIC). An inherent limitation of common AST practices is the difficulty of accurately recapitulating the in vivo bacterial growth environment on the lab bench. For example, in the complex environment of the lung, microbiota encounter a dynamic chemical milieu shaped by both host and microbial processes that varies dramatically from laboratory monoculture. Replicating the in vivo microbiota of the lungs and other sites of infection throughout the body is particularly important for understanding the role of polymicrobial community interactions on disease development and efficacy of antibiotic treatments. Accordingly, there remains a need in the art for improved methods of assessing antibiotic efficacy in view of polymicrobial community interactions and for improved therapeutic regimens for diseases caused by or associated with polymicrobial infections.

### SUMMARY OF THE INVENTION

Provided herein are methods and compositions for community-based antibiotic screening for use in treating polymicrobial infections. The methods and compositions find utility in selecting antibiotics and antibiotic combinations for treating diseases and conditions cuased by or associated with polymicrobial infections such as cystic fibrosis, chronic obstructive pulmonary disease, and chronic sinus infections. The methods and compositions of this disclosure are advantageous in the sense that it tests for antibiotic effectiveness on a community of microbes rather than one, more accurately reflect in vivo disease environments, and enable the identification of antimicrobial therapeutic(s) when the primary bacterial strain shows antibiotic resistance.

In one aspect, the disclosure provides a method for identifying an antibiotic suitable for treating polymicrobial infection, the method comprising: (a) combining in one or more containers a biological sample from a patient with a polymicrobial infection, one or more antibiotics and a media with a nutrient source, wherein the biological sample comprises at least a first microorganism and a second microorganism; (b) incubating the one or more containers; and (c) detecting the growth of the one or more microorganism in the one or more containers, wherein the reduction or inhibition of growth of at least one of the first or second microorganism identifies the one or more antibiotics for treating the polymicrobial infection. In some aspects, the method further comprises (d) treating the patient with the one or more antibiotics identified in step (c).

In another aspect, provided herein is a method for predicting the efficacy of an antibiotic in treating a polymicrobial infection causing or associated with a disease or condition in a patient in need thereof. The method can comprise or consist essentially of obtaining a biological sample from the patient, wherein the biological sample contains at least a first microorganism and a second microorganism, and in some examples, wherein the growth of the first microorganism is affected by the second microorganism for growth; adding the biological sample to one or more containers comprising an antibiotic and a media with a nutrient source; incubating the container; and determining the growth of at least one microorganism in the container, thereby predicting the efficacy of the antibiotic in treating the polymicrobial infection causing or associated with the disease or condition. The first microorganism can be a pathogenic microorganism, including, for example, a pathogenic bacteria, that causes or contributes to the disease or condition. In some aspects, the first microorganism can be affected by metabolites excreted from the second microorganism for growth. The second microorganism can be a bacterium in a community, and can be an anaerobic bacteria, an aerobic bacteria or a mixture.

In some aspects, the method further comprises isolating a community of microorganisms, including, for example, a community of bacteria, from a first portion of the sample to form a culture, wherein the community of microorganisms comprise the second microorganism; isolating one or more species of pathogenic microorganisms (e.g., pathogenic bacteria) from a second portion of the sample, wherein the one or more species of pathogenic microorganism (e.g., bacteria, etc.) comprise the first microorganism; adding the community culture to the container comprising the media and antibiotic; and adding the pathogenic microorganism (e.g., bacteria, etc.) to the container to perform the incubation step.

In some aspects, the method can comprise predicting the efficacy of two or more of antibiotics, wherein the method comprises adding the biological sample to two or more containers, wherein each container comprises a different antibiotic. The method can further comprise homogenizing the biological sample prior to adding the sample to the one or more or two or more containers.

The method can further comprise isolating a community of bacteria from a first portion of the sample to form a community culture, wherein the community of bacteria comprise the second microorganism; isolating one or more species of pathogenic bacteria from a second portion of the sample, wherein the one or more species of pathogenic bacteria comprise the first microorganism; adding the community culture to the container comprising the media and antibiotic; and adding the pathogenic bacteria to the container to perform the incubation step. The method can further comprise determining the MIC of the antibiotic required to inhibit growth of the community culture and determining the MIC of the antibiotic required to inhibit growth of the pathogenic bacteria.

In another aspect, provided herein is a container comprising a homogenized biological sample obtained from a subject obtained from a fluid, tissue, or wound containing a bacterial infection, wherein the biological sample comprises at least a first microorganism and a second microorganism, wherein growth of the first microorganism is affected by the presence of the second microorganism, wherein the container further comprises an antibiotic and a single nutrient source that reflects the environment of the polymicrobial infection. The container can be present in an array of containers. The container is a well in a multi-well plate. The nutrient source can be selected from the group consisting of mucin, amino acids, a carbohydrate, a sugar, lipid, nucleic acids, and collagen.

In another aspect, provided herein is a kit for determining the efficacy of multiple antibiotics in treating a disease or condition in a patient in need thereof, the kit comprising: an array of containers, wherein each container in the array comprises media, wherein the array of containers comprises control containers with no antibiotic and test containers, wherein each test container comprises one of the multiple antibiotics at varying concentrations; and instructions for adding a biological sample obtained from a patient to each container in the array and incubating the array to determine the efficacy of each antibiotic in treating the disease or condition.

In a further aspect, provided herein is a high throughput method for determining a suitable antibiotic for treatment of a disease or condition in a subject in need thereof, the method comprising obtaining a biological sample from the patient; adding a portion of the biological sample to each container in an array of containers, wherein each container in the array comprises an antibiotic and a media containing a nutrient source that reflects the environment of the biological sample, incubating the containers; and performing PCR to determine the growth of one or more selected bacteria in the container, thereby predicting the efficacy of the antibiotic in treating the polymicrobial infection.

In another aspect, provided herein is a high throughput method for screening small molecules, the method comprising obtaining a biological sample from a patient, wherein the biological sample is obtained from a fluid, tissue, or wound containing a bacterial infection; adding a portion of the biological sample to each container in an array of containers, wherein each container in the array comprises a small molecule and a media containing a nutrient source that reflects the environment of the biological sample; incubating the containers; and performing PCR to determine the growth of one or more bacteria in the container, thereby identifying small molecules that effectively inhibit growth of the bacteria in the environment of the infection.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A-1B illustrate (A) a mucin-based cross-feeding model of CF microbiota and (B) analysis of the “weakest link” phenomenon using CF sputum-derived cross-feeding communities. (A) P. aeruginosa (upper) is grown in soft-agar without a bioavailable nutrient source. Anaerobes (lower) are grown with mucin as a sole carbon source, liberating smaller metabolites for P. aeruginosa growth. In turn, P. aeruginosa creates an oxygen gradient allowing anaerobes to thrive. Under these co-operative culture conditions, inhibition of the anaerobic subpopulation with antibiotics to which P. aeruginosa is intrinsically resistant constrains overall community growth. (B) MICs of common antibiotics were determined for both P. aeruginosa and anaerobic consortia derived from patient samples. The effect of metabolic cross-feeding between subpopulations on each antibiotic is then determined using the mucin co-culture model.

FIGS. 2A-2C demonstrate antibiotic susceptibilities of CF-derived bacterial pathogens are impacted by co-operative community interactions. Minimum inhibitory concentrations (MICs) of six antibiotics were determined for (A) P. aeruginosa, and (B) anaerobic bacterial enrichments isolated from CF sputum. Grey and black bars represent predicted growth/no growth, respectively, at the concentration of antibiotic used in the community culture assay (C). Daggers indicate bacterial growth at the highest concentration of antibiotic tested. C, qPCR was used to quantify growth of sputum bacterial communities from which P. aeruginosa/anaerobes were derived in the presence of each antibiotic. Growth is expressed as % 16S rRNA gene copy numbers relative to an untreated control. Community growth was considered impaired if total 16S gene copies were less than 50% of the control. Assays were performed in triplicate for each sample/antibiotic combination.

FIG. 3 illustrates an exemplary workflow for screening clinical samples. Clinical samples were homogenized in a mock “sputum” environment and distributed among 1 mL glass culture tubes arrayed in deep-well 96-well plates. To each column of tubes was added a concentration gradient of a given antibiotic.

FIG. 4 demonstrates relative abundance of bacterial genera found in patient samples following antibiotic treatment. Bacterial DNA was extracted from stratified CF sputum cultures (agar plugs) treated with different classes of antibiotics (AZT, aztreonam; BAC, Bactrim; CIP, ciprofloxacin; DOX, doxycycline; LEV, levofloxacin; MET, metronidazole; TOB, tobramycin; ZOS, zosyn). Genomic DNA was subjected to 16S rRNA gene sequencing at the UMN Genomics Center and analyzed for bacterial community composition compared to an untreated control (NO ABX) and the original sputum sample. Colors represent the relative abundance of bacterial genera found within each sample and vary between treatments (demonstrating the community effect of antibiotic treatments).

FIG. 5 is a Table of the 20 most abundant genera of bacteria found in patients with sinusitis.

### DETAILED DESCRIPTION

In general, the present invention includes methods and compositions for community-based screening for the selection and administration of antibiotics and antibiotic combinations for treating polymicrobial infections causing or associated with diseases and conditions including, without limitation, cystic fibrosis, chronic obstructive pulmonary disease, and chronic sinus infections, among others. The methods and compositions of this disclosure are based at least in part on the inventor's development of assays and culture condition for study of polymicrobial infections in environments that more closely replicate the in vivo disease environment. Advantageously, the methods and compositions of this disclosure assess antibiotic effectiveness with respect to a community of microbes rather than a single microbial population, thus reflecting more accurately the in vivo disease environment and enabling the identification of beneficial antimicrobial therapeutics even when the primary bacterial strain shows antibiotic resistance. Without being bound to any particular mechanism or mode of action, it is believed that the identification of obligate or facultative relationships among microbial communities in a disease environment (e.g., bacterial community members in an airway) can be instructive to select antimicrobial agents to combat the disease. This strategy is particularly relevant for cases in which canonical pathogens (e.g., Pseudomonas aeruginosa) harbor intrinsic clinical resistance to multiple antibiotics. In particular, the methods permit indirect suppression of drug resistant pathogens such as Pseudomonas aeruginosa by identifying and inhibiting co-colonizing bacteria that stimulate the growth (through cross-feeding), virulence, or antibiotic resistance of the pathogen.

### Methods

The present disclosure provides methods of microbial community-based screening for identifying antimicrobials for use in treating polymicrobial infections. The methods have utility in selecting antibiotics and antibiotic combinations for treating polymicrobial infections that cause or are associated with diseases and conditions, and the identified antibiotics can be used to treat the subject or patient having such polymicrobial infections. The methods are advantageous in testing for antibiotic or combinations effective on a community of microbes rather than one microbe, more accurately reflect in vivo disease environments, and enable the identification of antimicrobial therapeutic even for polymicrobial infections where one of the primary bacterial strain shows antibiotic resistance.

In one embodiment, the disclosure provides a method for identifying an antibiotic suitable for treating polymicrobial infection, the method comprising: (a) combining in one or more containers a biological sample from a subject or patient with a polymicrobial infection, one or more antibiotics, and a media with a nutrient source, wherein the biological sample comprises at least a first microorganism and a second microorganism; (b) incubating the one or more containers; and (c) detecting the growth of the one or more microorganism in the one or more containers, wherein the inhibition or reduction of growth of at least one of the first or second microorganisms identifies the one or more antibiotics for use in treating the polymicrobial infection. The identified one or more antibiotic can then be used to treat the subject or patient in need thereof having the polymicrobial infection, as described in more detail below.

In another embodiment, provided herein is a method for predicting the efficacy of an antibiotic in treating a polymicrobial infection that causes or is associated with a disease or condition in a patient in need thereof. The method can comprise or consist essentially of obtaining a biological sample from the patient, wherein the biological sample contains at least a first microorganism and a second microorganism; adding the biological sample to one or more containers comprising an antibiotic and a media with a nutrient source that reflects the environment of the biological sample; incubating the container; and determining the growth of the first microorganism in the container, thereby predicting the efficacy of the antibiotic in treating the disease or condition.

In some cases, the first microorganism is a pathogenic bacteria that causes or contributes to the disease or condition. In some cases, the second microorganism is a bacterium in a community of bacteria. Preferably, the first microorganism is affected by metabolites excreted from the second microorganism for growth. For example, growth of the first microorganism can be promoted or hindered by a metabolite excreted from the second microorganism. In some examples, the community of bacteria is an anaerobic, an aerobic or mixed community of bacteria, and thus, in some examples, the second bacteria is an anaerobic or aerobic bacteria.

The term polymicrobial infection refers to an infection by two or more microorganisms, which can be, in some examples, two or more different bacterial species. The term microbial organism can be used interchangeably with microorganism.

In some embodiments, the one or more microbial organisms are pathogenic microorganisms, and in some examples, both the first and the second microorganism may be pathogenic organism. This in some embodiments, the polymicrobial infection can comprise the presence of infection with two or more species of pathogenic bacteria (e.g., the first microorganism and second microorganism are both pathogenic bacteria).

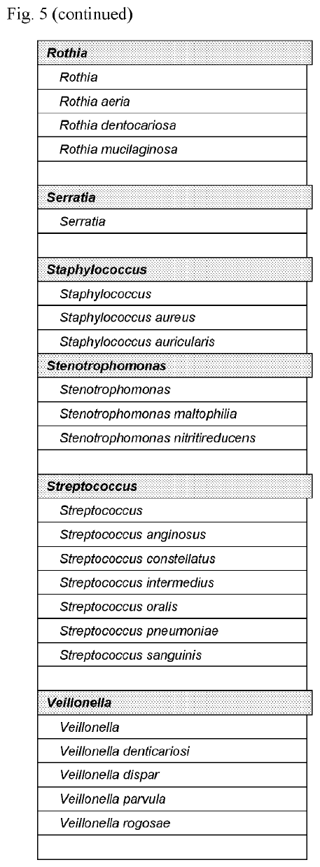

In some embodiments, only one of the microorganisms is a pathogenic organism. In some embodiments, the one or more of the microorganism may be a microorganism that is prone to antibiotic resistant or is antibiotic resistant. Microorganisms often responsible for healthcare-associated infections and prone to antibiotic resistance include, without limitation, methicillin-resistant Staphylococcus aureus (MRSA), Staphylococcus aureus, Enterococcus spp., Enterobacteriaceae (e.g., Escherichia coli, Enterobacter cloacae, Enterobacter aerogenes; Serratia marcesens, Klebsiella pneumoniae, Klebsiella oxytoca, Proteus mirabilis, Proteus vulgaris); Pseudomonas aeruginosa, and Acinetobacter spp., Streptococcus pneumoniae, Staphylococcus epidermidis, Hemophilus influenza; Helicobacter pylori, Salmonella typhimurium, Salmonella typhi, Salmonella paratyphi, E. coli H7:0157, E. coli MG1655, Shigella spp., Neisseria gonorrhoeae, Neisseria meningiditis; anaerobic organisms such as Bacteroides fragilis, Propionibacterium acnes, and Clostridium difficile; organisms of biothreat concern (e.g., Bacillus anthracis; Brucella abortus; Brucella melintensis; Brucella suis; Burkholderia mallei, Burkholderia pseudomallei; Francisella tularensis; Yersinia pestis); mycobacteria such as Mycobacterium tuberculosis complex; M. avium-intracellulare complex; M. kansasii; rapid-growing mycobacteria such as M. fortuitum; M. chelonae; M. abscessus; drug-resistant Candida albicans, Candida auris, and other Candida species; Cryptococcus neoformans and Cryptococcus gattii; unicellular and filamentous fungi such as Acinetobacter baumannii; Issatchenkia orientalis (I. orientalis), Saccharomyces cerevisiae (S. cerevisiae), Candida parapsilosis (C. parapsilosis), Candida neoformans, Candida gattii and Aspergillus fumigatus; and parasites such as Giardia lamblia and Entamoeba histolytica. Thus, in some embodiments, the biological sample may comprise a first microorganism is a pathogenic microorganism known in the art or described above or herein. Additional exemplary first microorganisms found in the biological sample can include, without limitation, a bacterial species selected from a bacterial genus including, for example, Pseudomonas, Staphylococcus, Haemophilus, Stenotrophomonas, Streptococcus, Mycobacterium, Enterococcus, Porphyromonas, Fusobacterium, Prevotella, Achromobacter, Veillonella, Rothia, Anaerococcus, Bacteroides, Corynebacterium, Cutibacterium, Finegoldia, Fusobacterium, Klebsiella, Lawsonella, Moraxella, Neisseria, and Serratia. Suitable microorganisms within these genuses are known and include, but are not limited to, for example, Pseudomonas aeruginosa, Pseudomonas fluorescens, Staphylococcus aureus, Staphylococcus epidermidis, Staphylococcus auricularis, Haemophilus influenza, Haemophilus parainfluenza, Stenotrophomonas maltophilia, Stenotrophomonas nitritireducens, Streptococcus pneumonia, Streptococcus milleri, Streptococcus anginosus, Streptococcus constellatus, Streptococcus intermedius, Streptococcus oralis, Streptococcus sanguinis, Mycobacterium abscessus, Mycobacterium avium, Mycobacterium intracellulare, Enterococcus faecalis, Porphyromonas gingivalis, Porphyromonas endodontalis, Porphyromonas pasteri, Fusobacterium nucleatum, Fusobacterium periodonticum, Prevotella baroniae, Prevotella bivia, Prevotella buccalis, Prevotella conceptionensis, Prevotella dentalis, Prevotella denticola, Prevotella disiens, Prevotella histicola, Prevotella intermedia, Prevotella loescheii, Prevotella melaninogenica, Prevotella nanceiensis, Prevotella nigrescens, Prevotella oris, Prevotella pallens, Prevotella salivae, Prevotella shahii, Prevotella timonensis, Prevotella melaninogenica, Prevotella veroralis, Achromobacter xylosoxidans, Veillonella parvula, Veillonella denticariosi, Veillonella dispar, Veillonella rogosae, Rothia mucilaginosa, Rothia aeria, Rothia dentocariosa, Anaerococcus nagyae, Anaerococcus octavius, Anaerococcus provenciensis, Bacteroides acidifaciens, Corynebacterium amycolatum, Corynebacterium appendicis, Corynebacterium kroppenstedtii, Corynebacterium matruchotii, Corynebacterium pseudodiphtheriticum, Corynebacterium tuberculostearicum, Cutibacterium acnes, Cutibacterium avidum, Cutibacterium granulosum, Finegoldia magna, Fusobacterium nucleatum, Fusobacterium periodonticum, Klebsiella pneumoniae, Klebsiella oxytoca, Lawsonella clevelandensis, Moraxella nonliquefaciens, Neisseria flavescens Neisseria oralis, Neisseria polysaccharea, Serratia and Burkholderia cepacia complex (BCC; also known as B. cepacia), among others. Suitable bacteria are also listed in FIG. 5 which were identified in samples from patients with chronic sinusitis.

The second microorganism can be a microorganism in a community of microorganisms, for example, in some embodiments, the second microorganism is a bacterium in a community of bacteria. The second microorganism may be a microorganism from the community in which the first microorganism is found. In some examples, the community of microorganisms is a community of bacteria and may be a community of aerobic bacteria, anaerobic bacteria or mixtures thereof. In some embodiments, the first microorganism is a pathogenic microorganism, for example, a pathogenic bacteria. In some embodiments, the second microorganism is a pathogenic microorganism, for example, a pathogenic bacteria. In some embodiments, the second microorganism is a bacteria, and the bacteria is an aerobic or anaerobic bacteria, and may be selected from the group described above. Not to be bound by any theory, in some embodiments, it is believed that the community microorganisms (e.g., bacteria, etc.), including the second microorganism, may provide additional factors to the microenvironment that alter the growth of the first microorganism (including in examples where the first bacteria is a pathogenic or antibiotic resistant bacteria), and thereby identifying antibiotics by the method described herein can inhibit microbial (or bacterial) growth of the first microorganism by regulating the factors provided by the community of microorganisms (e.g., by inhibiting growth of the second microorganism and/or community microorganism or bacteria, and therefore altering the factors excreted by such microorganisms or bacteria).

The polymicrobial infection can cause or be associated with a disease or condition. Diseases or conditions for which the methods of this disclosure are particularly well suited include, without limitation, cystic fibrosis (CF), chronic obstructive pulmonary disorder (COPD), chronic sinusitis, a gastrointestinal infection, a chronic wound, septicemia, a urinary tract infection, or a renal infection. Other diseases and conditions for which the methods of this disclosure are particularly well suited include, without limitation, abscesses, AIDS-related opportunistic infections, conjunctivitis, gastroenteritis, hepatitis, multiple sclerosis, otitis media, periodontal diseases, osteomyelitis, burn wounds, urinary tract infection, vaginitis, medical device-related infection, pneumonia, keratitis, peritonitis, diabetic foot wound infections, etc. One skilled in the art is able to identify suitable samples and conditions for which the methods of the present disclosure are suitable.

Any appropriate method of determining growth of the first microorganism can be performed. Suitable techniques include, without limitation, visual inspection for turbidity in the container, optical density measurement, colorimetric assays (e.g., pH, redox, and chemical indicator dyes), disc diffusion agar plate methods, optical imaging, quantitating a molecular, biochemical or metabolic marker of the bacteria, quantitating 16S rRNA, and performing PCR. Microorganism growth can be determined, for example, by measuring cell density of the cultures by optical density. In some cases, quantitative PCR (qPCR) is used to quantify growth of microorganisms and/or microorganism communities in the presence or absence of an antibiotic. Growth can be expressed, for example, as a percentage of 16S rRNA gene copy numbers relative to an untreated control. In some cases, community growth is considered to be impaired if the total number of 16S gene copies is less than 50% of the control.

Any appropriate biological sample can be obtained from a subject for the methods of this disclosure. Exemplary biological samples include, without limitation, sputum, saliva, nasal fluid, respiratory secretions, fluid from a wound (e.g., wound exudate), human tissues (e.g., lung) or a laboratory culture thereof, blood, feces, and urine, other body fluids, implanted medical devices, dental plaque, vaginal mucus, inner ear exudate, or combinations thereof.

The biological sample may be obtained from the following: patients with debilitated immune systems, patients with septicemia, sputum samples from patients with pneumonia, sputum samples from patients with cystic fibrosis, endotracheal samples from intubating patients under intensive care, samples from urinary catheters, samples from wounds, and include any suitable sample obtained from a patient suffering from cystic fibrosis.

As used herein, the term “subject” refers to an individual having, suspected of having, or susceptible to having a disease or condition associated with or caused by a polymicrobial infection (e.g., a disease or condition associated with). By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on. In a preferred embodiment, the subject is a human having a polymicrobial infection.

Preferably, the biological sample is added to one or more containers comprising an antibiotic, a culture medium, and a nutrient source. The nutrient source can be selected by one skilled in the art and preferably mimics the environment from which the biological sample was obtained. Suitable nutrient sources are known and understood by one skilled in the art, as described more herein.

As described herein, the culture medium preferably comprises a nutrient source that reflects the environment of the biological sample. Any appropriate culture medium can be used for the methods of this disclosure. In some cases, the medium contains a single carbon source. In some cases, the medium contains one or more nutrient sources such as, for example, mucin, amino acids, a carbohydrate, a sugar, lipids, nucleic acids, and collagen. For example, P. aeruginosa grows well on LB broth, but can also utilize a wide range of compounds as sole carbon and/or nitrogen sources. Accordingly, various chemically defined medium formulations such as MOPS (3-(N-Morpholino) Propane-Sulfonic Acid) medium, M9, or M6 can be used. The amount of each component can be determined as appropriate depending on the type of cell(s) to be cultured, the type of biological sample, the relevant microorganisms, and the like. When a chemically defined minimal medium is used, the medium can be supplemented with one or more selected nutrient sources as necessary. It may be advantageous in some cases to use a culture medium that is permissive to growth of only certain bacterial types (e.g., gram negative or gram positive) or certain strains of bacteria (e.g., resistant to a particular antibiotic). Such a system can be used to identify the presence or absence of a microbiological infection, such as a bacterial infection, in a subject.

In some embodiments, the culture medium can further comprises a gelling agent. Suitable gelling agents are known in the art that allow for microorganism growth within the culture. Not to be bound by any theory, but the gelling agent my further thicken or solidify the media to provide a suitable environment for allowing the one or more microorganism to grow. Suitable gelling agents include, but are not limited to, for example, polymeric gelling agents (e.g., carbomers), cellulose-based gelling agents (e.g., hydroxypropyl cellulose and the like), natural gelling agents (e.g., xanthan gum, guar gum, gellan, pectin, gelatin, agar, and the like). Suitable amounts of the gelling agent can be readily determined by one skilled in the art and may comprise from about 0.01% to about 5%, preferably from about 0.01% to about 2% of the gelling agent (e.g., 0.01%, 0.05%, 0.1%, 0.5%, 0.75%, 1%, 1.25%, etc). For example, in one embodiment, the culture medium can comprises agar. As used herein, the term “agar” refers to any agar base that is suitable for the growth of microorganisms and encompasses any agar bases that contains basic ingredients which will support the growth of microorganisms when such base is supplemented with a suitable growth supplement. In some cases, the agar is agarose. Other agars include, without limitation, blood agar, Endo agar, Noble agar, and cetrimide agar. In some cases, agar is present at a concentration of about 0.01% to about 2% (e.g., about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%). In certain cases, agar is present at a concentration of about 1%. As used with regard to the concentration of gelling agent in the culture medium, the term “about” can include a variance of 1% in either direction, even though the target concentration is the stated concentration (e.g., “about 10%” can include 9% and 11%).

As described herein, the methods make it possible to predict the efficacy of an antibiotic or combination of antibiotics in treating the disease or condition based on polymicrobial community interactions in an in vitro environment that replicates the endogenous disease environment. The methods allow for the identification of one or more antibiotics that can be used for treating the polymicrobial infection.

Suitable antimicrobial or antibiotics that can be used in the methods described herein include antibiotics that are known in the art for treating one or more types of microbial infections. Suitable classes of antimicrobials and antibiotics include, but are not limited to, for example, an aminoglycoside, a carbapenem, a cephalosporins, a fluoroquinolone, a glycopeptide, a lipoglycopeptide, a macrolide, a monobactam, an oxazolidinone, a penicillin, a polypeptide, a rifamycin, a chloramphenicol, a quinolones, a sulfonamide, a streptogramin, a tetracycline, a beta-lactam antibiotic, an ansamycin, a streptagramin, chloramphenicol, clindamycin, daptomycin, fosfomycin, lefamulin, metronidazole, mupirocin, nitrofurantoin, and tigecycline or a combination thereof. Antiboitics include ones known or developed that may or may not fall into these categories but can be used for treating a microbial infection. In some cases, a combination of one or more antibiotics may be used in the screen to be able to inhibit the polymicrobial infection.

Appropriate antibiotics for the methods of this disclosure include, without limitation, penicillin, amoxicillin, cephalosporin, cephalexin, streptomycin, neomycin, kanamycin, paromomycin, vancomycin, teicoplanin, trovafloxacin, linezolid, posizolid, tedizolid, cycloserine, prontosil, sulfanilamide, sulfadiazine, sulfisoxasole, tetracycline, limecycline, oxytetracycline, erythromycin, clarithromycyin, chloramphenicol, clindamycin, fosfomycin, lefamulin, metronidazole, mupirocin, nitrofurantoin. tigecycline, azithromycin, geldanamycin, rifamycin, naphthomycin, pristinamycin, daptomycin, sufactin, tobramycin, aztreonam, levofloxacin, piperacillin, tazobactam, trimethoprim-sulfamethoxazole, metronidazole, ciprofloxin, doxycycline, meropenem, clindamycin, chloramphenicol, cefoxitin, ticarcillin, ampicillin, clavulanic acid, sulbactam, co-trimoxazole, colistin, dicloxacillin, amoxicillin, ceftazidime, amikacin, gentamicin, netilmicin, ansamycins, herbimycin, rifaximin, carbacephem, loracarbef, carbapenems, ertapenem, doripenem, imipenem/cilastatin, cephalosporins (first-fifth generation), cefadroxil, cefazolin, cephradine, cephapirin, cephalothin, cefalexin, cefaclor, cefoxitin, cefotetan, cefamandole, cefmetazole, cefonicid, loracarbef, cefprozil, cefuroxime, cefixime (antagonistic with chloramphenicol), cefdinir, cefditoren, cefoperazone, cefotaxime, cefpodoxime, ceftibuten, ceftizoxime, moxalactam, ceftriaxone, cefepime, ceftaroline fosamil, ceftobiprole, glycopeptides, teicoplanin, telavancin, dalbavancin, oritavancin, lincomycin, lipopeptide, clarithromycin, roxithromycin, telithromycin, spiramycin, fidaxomicin, nitrofurans. furazolidone, radezolid, torezolid, azlocillin, flucloxacillin, mezlocillin, methicillin, nafcillin, oxacillin, temocillin, penicillin combinations, amoxicillin/clavulanate, piperacillin/tazobactam, ticarcillin/clavulanate, bacitracin, polymyxin b, enoxacin, gatifloxacin, gemifloxacin, lomefloxacin, moxifloxacin, nadifloxacin, nalidixic acid, norfloxacin, ofloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfasalazine, trimethoprim-sulfamethoxazole (co-trimoxazole) (tmp-smx), sulfonamidochrysoidine (archaic), demeclocycline, metacycline, minocycline, clofazimine, dapsone, capreomycin, ethambutol(bs), ethionamide, isoniazid, pyrazinamide, rifampicin, rifabutin, rifapentine, arsphenamine, chloramphenicol(bs), fosfomycin, fusidic acid, platensimycin, quinupristin/dalfopristin, thiamphenicol, tigecycline(bs), tinidazole, and combinations thereof, among others. Suitably, the method can be applied to newly developed or discovered antimicrobials as well as those known in the art, and thus are not limited to those described herein. The one or more antibiotics can also be tested at one or more different concentrations, and in different combinations of the one or more antibiotics to identify antibiotics that can work for treatment of polymicrobial infections.

In some cases, the method comprises incubating the container at a relevant body temperature for at least 24 hours prior to determining the growth of the at least one organism, (in some embodiments, the at least one organism is the first microorganism). Body temperature is commonly in the range of 36° C.-38° C., but other temperatures will be appropriate based on the relevant microorganism(s) and body tissue or source of biological sample. In some cases, microorganism growth can be determined, for example, by measuring cell density of the cultures by optical density.

In some cases, portions of a biological sample are added to two or more containers, where the containers comprise different concentrations of a single antibiotic. Preferably, the container comprise the antibiotic at a concentration between about 0.01 μg/mL to about 1000 μg/mL, but other antibiotic concentrations may be used. In some embodiments, two or more antibiotics can be added to a single container.

In some cases, the method comprises predicting the efficacy of or identify two or more of antibiotics that can inhibit polymicrobial infections. In such cases, the method comprises adding the biological sample to two or more containers, where each container contains a different antibiotic.

It will advantageous in some cases for the method to further comprise homogenizing the biological sample prior to adding the sample to the one or more containers.

In some cases, the method further comprises the following steps: isolating a community of bacteria from a first portion of the biological sample to form a community culture, where the community of bacteria comprise the second microorganism; isolating one or more species of pathogenic bacteria from a second portion of the biological sample, where the one or more species of pathogenic bacteria comprise the first microorganism; adding the community culture to the container comprising the media and antibiotic; and adding the pathogenic bacteria to the container to perform the incubation step.

In some cases, the method additionally comprises the following steps: determining the minimum inhibitory concentration (MIC) of the antibiotic required to inhibit growth of a community culture and determining the MIC of the antibiotic required to inhibit growth of the pathogenic bacteria. As used herein, a term “minimum inhibitory concentration” or “MIC” refers to the lowest concentration of active agent that inhibits growth of a microorganism. MIC determinations may be made according to processes known in the art. The MIC may be expressed as a particular concentration value or as a range of concentrations. In some cases, the first or second microorganism is anaerobic. In some cases, the first or second microorganism is aerobic.

In another aspect, provided herein is a high throughput method for determining a suitable antibiotic for treatment of a disease or condition in a subject in need thereof. In preferred embodiments, the method comprises the following steps: obtaining a biological sample from the patient; adding a portion of the biological sample to each container in an array of containers, wherein each container in the array comprises an antibiotic and a media containing a nutrient source that reflects the environment of the biological sample; incubating the containers; and performing PCR to determine the growth of one or more selected bacteria in the container, thereby predicting the efficacy of the antibiotic in treating the disease or condition. In some cases, the array of containers comprises an array of wells in a multi-well plate or an array of culture tubes. Preferably, the biological sample contains at least a first microorganism and a second microorganism, where the first microorganism is affected by the presence of the second microorganism for growth.

As used herein, the terms “treating,” “treat,” and “treatment” refer to the management and care of a patient for the purpose of combating the disease, condition, or disorder. The terms embrace both preventative, i.e., prophylactic, and palliative treatments. In some cases, the term “treated” refers to any beneficial effect on progression of a disease or condition. Beneficial effects can include reversing, alleviating, inhibiting the progress of, preventing, or reducing the likelihood of the disease or condition to which the term applies or one or more symptoms or manifestations of such a disease or condition. Where the disease or condition is polymicrobial infection, treating can refer to the management and care of a patient for the purpose of combating the infection, and can include reversing, alleviating, inhibiting the progress of, preventing, reducing the size of, or reducing the likelihood of, or lessening the severity of any aspect of the polymicrobial infection or infection-associated condition. In some cases, the treating inhibiting the growth of the one or more bacterial infections within the polymicrobial infection.

In some cases, the method comprises administering one or more antibiotics identified according to the methods of this disclosure to be effective against the subject's disease or condition.

In another aspect, provided herein is a high throughput method for screening small molecules. In preferred embodiments, the method comprises the following steps: adding a portion of a biological sample obtained from a subject to each container in an array of containers, where each container in the array comprises a test small molecule and a media containing a nutrient source; incubating the containers; and performing PCR to determine the growth of one or more bacteria in the container, thereby identifying small molecules that effectively inhibit growth of the bacteria. In some embodiments, the method further comprises obtaining a biological sample from a patient, where the biological sample is obtained from a fluid, tissue, or wound containing a bacterial infection. Preferably, the biological sample contains at least a first microorganism and a second microorganism. In some embodiments, where the first microorganism is affected by the presence of the second microorganism for growth. In some cases, the array of containers comprises an array of well in a multi-well plate. In some cases, the method comprises incubating the biological sample in the presence of culture medium and small molecule for a predetermined length of time; and then screening cultures resulting from the incubation for evidence of growth inhibition relative to a control that lacks the test small molecule. In some cases, screening comprises measuring relative growth of a microorganism culture derived from the biological sample and a control sample. Suitable methods of detecting growth are described above.

Compositions

In another aspect, provided herein is a container comprising a homogenized biological sample obtained from a subject. Preferably, the biological sample is obtained from a fluid, tissue, or wound containing a bacterial infection. More preferably, the biological sample comprises at least a first microorganism and a second microorganism, where the first microorganism is affected by the presence of the second microorganism for growth. Exemplary microorganisms found in the biological sample include, without limitation, the bacteria described above in the methods. In some cases, the biological sample comprises a plurality of bacteria, in some embodiments; the plurality of bacteria can be a plurality of anaerobic bacteria, a plurality of aerobic bacteria or mixtures thereof.

In some cases, the container is present in an array of containers. For example, the container can be a well in a multi-well plate. In some cases, the array of containers comprises an array of wells in a multi-well plate or an array of culture tubes.

In some cases, the container further comprises an antibiotic and a single nutrient source that reflects an in situ environment of the bacterial infection. In some cases, the nutrient source is selected from the group consisting of mucin, amino acids, a carbohydrate, a sugar, lipid, nucleic acids, and collagen.

In a further aspect, provided herein is a kit for determining efficacy of multiple antibiotics in treating a polymicrobial infection causing or associated with a disease or condition in a patient in need thereof, the kit comprising: an array of containers, where each container in the array comprises a culture medium. In some cases, the array of containers comprises one or more control containers that contain no antibiotic. In addition, the array of containers comprises one or more test containers, where each test container comprises one of the multiple antibiotics at a predetermined concentration. In some cases, the test containers collective comprise one of the antibiotics at varying concentrations. In some embodiments, the kit comprises a combination of two or more antibiotics at the same or differing concentrations. In some cases, the kit further comprises instructions for adding a biological sample obtained from a patient to each container in the array and incubating the array to determine the efficacy of each antibiotic in treating the disease or condition.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

It should also be understood that, unless clearly indicated to the contrary, in any methods claimed herein that include more than one step or act, the order of the steps or acts of the method is not necessarily limited to the order in which the steps or acts of the method are recited.

The present invention has been described in terms of one or more preferred embodiments, and it should be appreciated that many equivalents, alternatives, variations, and modifications, aside from those expressly stated, are possible and within the scope of the invention. The invention will be more fully understood upon consideration of the following non-limiting Examples. The invention will be described in greater detail by way of specific examples. The following examples are offered for illustrative purposes, and are not intended to limit the invention in any manner.

### Examples

The Examples in the following section demonstrate that, when patient-derived microbiota are cultured under mutualistic cross-feeding conditions or in the presence of co-colonizing bacteria, interactions between bacteria have profound impacts on the efficacy of CF therapies. These data suggest that control of chronic lung infections may be achieved by exploiting obligate or facultative relationships among airway bacterial community members. This strategy is particularly relevant for cases in which canonical pathogens (e.g., Pseudomonas aeruginosa) harbor intrinsic clinical resistance to multiple antibiotics.

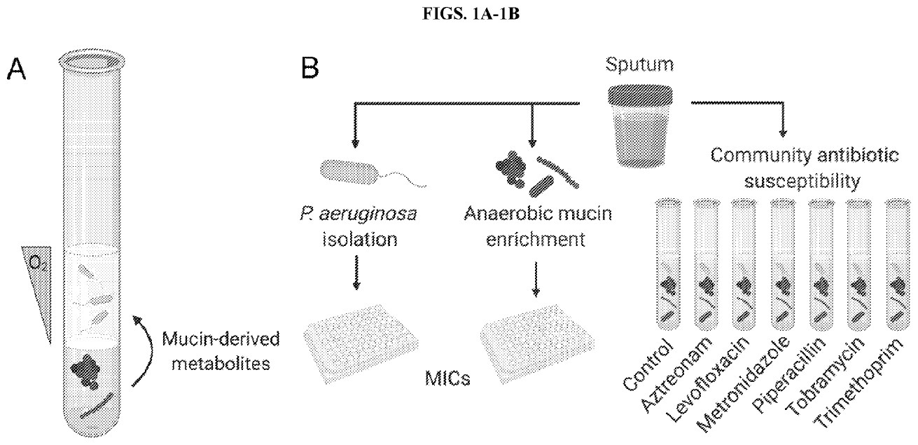

While interspecies interactions complicate medical management, co-operative relationships among CF microbiota may offer potential opportunities for development of new therapeutic strategies. For example, in microbial communities harboring multiple species that rely on one another for essential metabolites (termed ‘cross-feeding’), interdependence can render the community highly susceptible to failure relative to the same community members living independently. This was recently demonstrated using a synthetic, three-member consortium engineered to be mutualistic (co-operative) or competitive, depending on the nutritional environment (9). Under co-operative conditions, effective community tolerance (antibiotic concentration needed to inhibit overall community growth) was set by the least tolerant member (i.e., the “weakest link”). This phenomenon was also shown using a model co-operative CF bacterial community composed of P. aeruginosa and anaerobic bacteria capable of degrading mucins (host glycoproteins produced by the airway epithelium)(9, 10). When co-cultured on mucins as a sole carbon source, P. aeruginosa establishes an O₂ gradient allowing anaerobes to grow, which in turn liberate mucin-derived metabolites that P. aeruginosa is unable to obtain on its own (FIG. 1A). Under these co-culture conditions, despite the high intrinsic resistance of P. aeruginosa to ampicillin (determined by standard AST), ampicillin-inhibition of the anaerobic subpopulation constrained P. aeruginosa growth. These data suggest that targeting the weakest link among interdependent microbiota may be a viable strategy for inhibiting pathogen growth in vivo.

To further test this idea under clinically relevant conditions, sputum was collected from adult CF patients and isolated P. aeruginosa from each sample (FIG. 1B). Using a panel of widely used CF antibiotics (tobramycin, aztreonam, levofloxacin, piperacillin-tazobactam) and others lacking conventional activity against P. aeruginosa (trimethoprim-sulfamethoxazole, metronidazole), MICs for each isolate were determined under nutrient-rich conditions (akin to standard AST procedures). Concurrently, enrichment culturing was performed to isolate anaerobic mucin-degrading bacteria from each sample (10) and antibiotic MICs were similarly determined. Finally, small aliquots of remaining sputa were diluted in buffer and split between culture tubes containing mucin as the sole carbon source, 1% agarose, and one of the antibiotics listed above. Using these cultures in which P. aeruginosa and anaerobic mucin-degraders were co-dependent on mucin utilization, how co-operative growth affected antibiotic sensitivity relative to independent culture was interrogated.

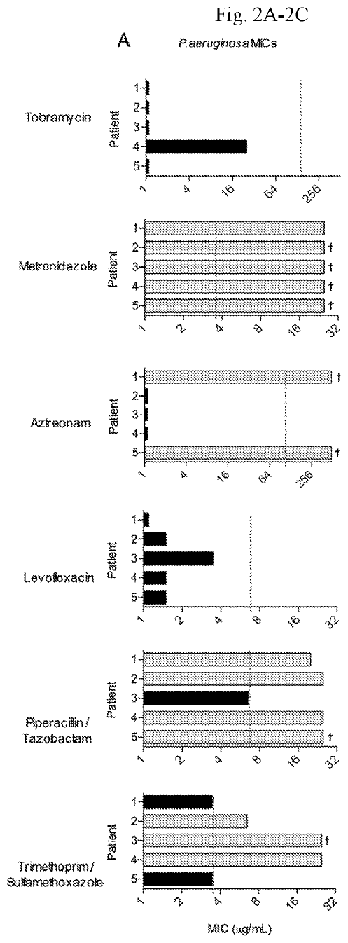

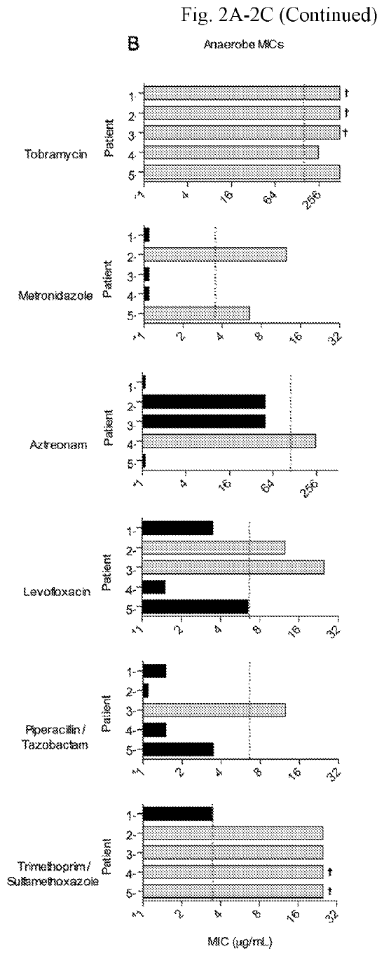

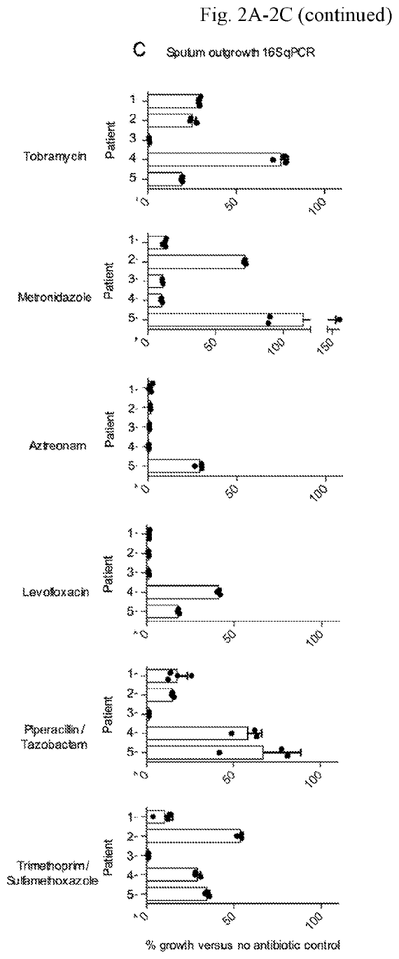

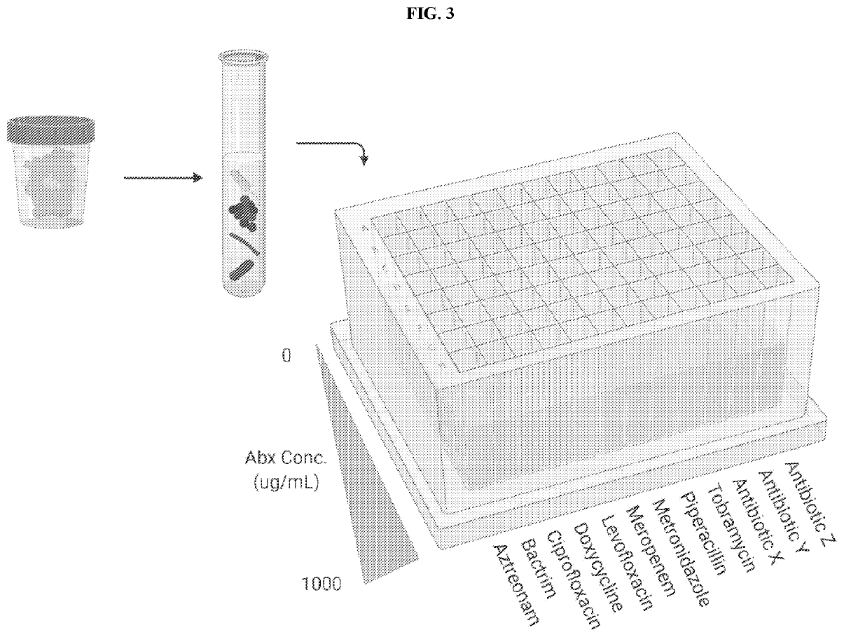

MICs of P. aeruginosa and anaerobic cultures are shown in FIGS. 2A and 2B, respectively. Tobramycin and levofloxacin were broadly effective against P. aeruginosa, while some isolates were resistant to one or more of aztreonam, piperacillin-tazobactam (P/T) and trimethoprim-sulfamethoxazole (T/S) (FIG. 2A). Metronidazole, known to have limited activity against aerobic bacteria, was ineffective. For the anaerobic enrichments (FIG. 2B), all compounds showed variable efficacy including those not recognized for anaerobic coverage (aztreonam, levofloxacin), with the exception of tobramycin to which all anaerobic enrichments demonstrated resistance. These data are consistent with previous work characterizing antibiotic efficacy against P. aeruginosa and anaerobic CF isolates (11, 12).

It was assessed whether co-operative community growth (see FIG. 1A) would affect antibiotic susceptibility. Specifically, we hypothesized that the sensitivity of the overall sputum bacterial community would be set by the least tolerant among P. aeruginosa and anaerobic subpopulations. To test this hypothesis, total 16S rRNA gene copy number was used to quantify community growth in response to each antibiotic relative to untreated controls (FIG. 2C). As proof-of-concept, we first tested two compounds with contrasting efficacy: (i) tobramycin, a common CF therapy with efficacy against P. aeruginosa, and (ii) metronidazole, an atypical CF antibiotic known for its anaerobic coverage. Both compounds showed a striking effect on co-operative cultures. Specifically, community growth was constrained if either P. aeruginosa or the anaerobic subpopulation was susceptible in monoculture. Conversely, in instances where both subpopulations demonstrated independent resistance to the concentration of antibiotic used in the co-culture assay, sputum community growth was also robust (e.g., metronidazole for patient 5).

For each of the other compounds tested (aztreonam, levofloxacin, P/T, and T/S), the presence of at least one antibiotic-susceptible population was largely predictive of community failure when treated with that same compound. For example, growth of two aztreonam-resistant P. aeruginosa isolates was restricted in co-culture, likely due to the aztreonam susceptibility of their anaerobic mutualists. However, notable exceptions were also observed, particularly for T/S which showed variable effects (i.e., co-cultures were unaffected despite a susceptible subpopulation). In two instances, community growth was surprisingly inhibited despite the predicted inefficacy of the antibiotic based on individual MICs (T/S for patients 3 and 4). As expected, no instances observed in which community growth resulted from a co-culture of P. aeruginosa and anaerobes that were both independently susceptible.

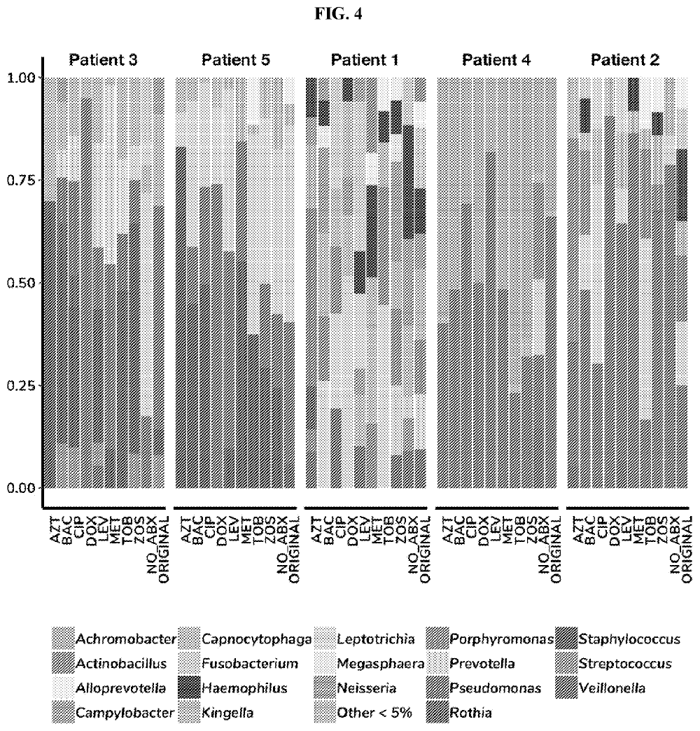

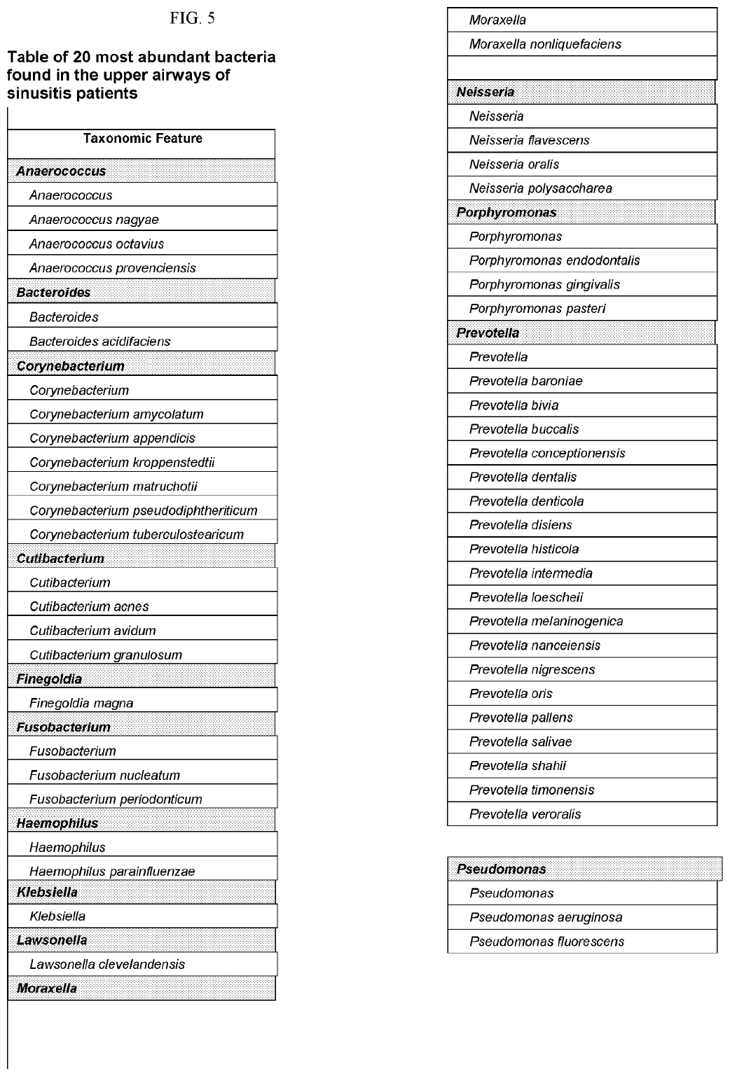

As shown in FIG. 4, antibiotic treatment of patient samples altered the relative abundance of different bacterial genera in the community. For these assays, bacterial DNA was extracted from stratified CF sputum cultures (agar plugs) treated with different classes of antibiotics (AZT, aztreonam; BAC, Bactrim; CIP, ciprofloxacin; DOX, doxycycline; LEV, levofloxacin; MET, metronidazole; TOB, tobramycin; ZOS, zosyn). Genomic DNA was subjected to 16S rRNA gene sequencing at the University of Minnesota Genomics Center and analyzed for bacterial community composition compared to an untreated (no antibiotic) control (“NO ABX”) and the original sputum sample. Colors represent the relative abundance of bacterial genera found within each sample and vary between treatments. These data demonstrate that antibiotic treatment not only affects the targeted pathogen (Pseudomonas aeruginosa) but also the bacteria community in which it is found. These data can be used to predict how well a patient will respond to a given antibiotic.

### DISCUSSION

There are several possible explanations for the observed variable effects. First, independent MICs were determined in a mixed broth culture, whereas co-operative growth was assayed in a spatially structured gel. It is known that spatial relationships within- and between-species can have profound effects on their physiology (13) and it is likely that bacterial distribution throughout the co-cultures was an important determinant of community antibiotic susceptibility. In addition, while the taxonomic composition of sputum microbiota was not determined, differences in community membership could explain disparities between patients for a given antibiotic. Specifically, different anaerobic species and other canonical pathogens excrete unique metabolites, degradative enzymes, and modify the growth environment in ways such that they can differentially affect antibiotic efficacy. Ongoing work is focused on how community membership, spatial structure and varying antibiotic concentrations affect the community susceptibility phenotypes described here.

These data demonstrate a clear community effect on antibiotic efficacy and underscore that pathogen susceptibility is defined not only by the intrinsic resistance of a target organism but also on the dynamics of its co-colonizers and growth environment in which they are found. It has been widely documented that antibiotic-sensitive bacteria can elude antibiotic killing in vivo as a result of co-existing antibiotic-resistant organisms (e.g. via production of extended-spectrum β-lactamases (8)). Similarly, P. aeruginosa and S. aureus have been shown to stimulate vancomycin resistance in Streptococcus anginosus via alteration of its cell wall (6). Such interactions are commonly put forth as explanations of why antibiotics fail clinically despite their demonstrated efficacy in vitro. The converse effect is demonstrated here: drug resistant pathogens can be indirectly suppressed by inhibiting their cross-feeding partners. In this context, identifying the weakest links among CF microbiota could improve upon standardized AST and the efficacy of therapies aimed at treating chronic lung disease. Moreover, these “off-target” effects may explain, at least in part, why some antibiotics predicted to fail can ultimately have beneficial effects on patients that receive them.

Given the diversity and nature of interspecies interactions in the CF airways, the weakest-link approach is broadly applicable beyond mucin-based cross-feeding. In theory, any bacterial community in which two or more members exchange metabolites, potentiate virulence, or rely on others for niche modification (e.g. oxygen consumption) could be targeted. For example, fermentation products (e.g., 2,3-butanedione) and cell wall fragments (N-acetyl-glucosamine) derived from Gram-positive bacteria significantly increase virulence and biofilm formation of P. aeruginosa (14, 15). Likewise, P. aeruginosa exhibits enhanced pathogenicity in various host models when co-cultured with oropharyngeal flora relative to P. aeruginosa infection alone (16, 17). As these interactions are more clearly defined and new community relationships are identified, novel therapies are likely to emerge. Such strategies may be critical for non-tuberculosis mycobacteria and other emerging pathogens whose innate multi-drug resistance poses a significant barrier to direct treatment.

Methods

Sputum collection. Sputum was collected from adult participants with CF and processed within 30 minutes of collection. Studies were approved by the UMN Institutional Review Board (#1511M8052).

Pseudomonas aeruginosa isolation. 100 μL of sputum was streaked onto Pseudomonas isolation agar and incubated at 37° C. for 72 hours. A single isolate from each sample was screened by PCR to confirm identity and stored in 30% glycerol at −80° C.

Anaerobic enrichment. Sputum was passed into a Coy anaerobic chamber (95% N₂, 5% CO₂). Equal volumes of minimal mucin medium (MMM) (10) and phosphate buffered saline (PBS) were then added followed by mechanical homogenization using 18-gauge needles. 100 of each sample was added to 5 mL of MMM containing 15 g/L purified porcine gastric mucin (10) and incubated at 37° C. for 72 h under anoxia to enrich for anaerobic mucin-degrading bacteria.

Antibiotic susceptibility testing. For anaerobic AST, 5 μL of enrichment culture was added to each well of microtiter plate containing MMM supplemented with gradients of seven antibiotics (aztreonam 0-500 μg/mL; doxycycline, 0-25 μg/mL; levofloxacin, 0-25 μg/mL; metronidazole, 0-25 μg/mL; piperacillin/tazobactam, 0-25 μg/mL; tobramycin, 0-500 μg/mL; trimethoprim/sulfamethoxazole, 0-25 μg/mL). Plates were wrapped in parafilm and incubated anaerobically at 37° C. for 72 h. For P. aeruginosa, isolates were grown in lysogeny broth (LB) to an OD₆₀₀ of 0.5. 5 μL was then added to a similar plate containing LB as the base medium and incubated aerobically at 37° C. for 24 h. Plates were resuspended by pipetting and read spectrophotometrically at OD₆₀₀ using a BioTek Synergy plate reader. MICs were determined based on the antibiotic concentration required to achieve a 90% reduction in culture density relative to antibiotic-free controls.

Co-culture. Under anaerobic conditions, 200 μL of homogenized sputum was placed into one of eight tubes to which a pre-determined concentration of each antibiotic was added (aztreonam, 100 μg/mL; doxycycline, 6.5 μg/mL; levofloxacin, 6.5 μg/mL; metronidazole, 3.5 μg/mL; piperacillin/tazobactam, 6.5 μg/mL; tobramycin, 500 μg/mL; trimethoprim/sulfamethoxazole, 3.5 μg/mL). Concentrations were selected based on prior MIC assays demonstrating variable efficacy against anaerobic populations. ‘No antibiotic and cell-free’ tubes were used as controls. 200 μL of molten 2% low melting point agarose in PBS was added to each tube and homogenized by pipetting. Each mixture was then added to 0.5 mL Pyrex culture tubes forming a gel upon cooling. Upon removal from the anaerobic chamber, tubes were incubated at 37° C. for 72 h and frozen at −80° C.

DNA extraction. Agar plugs were removed from the culture tubes, transferred to a Matrix E Lysing Tubes (MP Biomedicals) and two volumes of lysis buffer were added. Tubes were vortexed on a bead beater for 2×30 s, followed by centrifugation at 9,500×g for 1 min. Supernatant was transferred to a new tube, incubated at 75° C. for 10 min and returned to room temperature. Lysozyme and RNAse A were then added to final concentrations of 0.2 mg/mL and 50 μg/mL, respectively, and incubated for 1 h at 37° C. 1004, of 10% SDS and proteinase K (2 mg/mL) were added and incubated overnight at 55° C.

DNA was extracted by adding one volume of phenol:chloroform:iosoamyl alcohol (25:24:1) and vortexing to emulsify. Samples were centrifuged at 21,000×g for 20 min and the upper aqueous layer transferred to a new tube. One volume of chloroform:IAA was then added followed by centrifugation at 21,000×g for 15 min. The upper layer was transferred to a new tube. 3M sodium acetate (pH 5.2) was added and mixed gently, followed by the addition of 1 volume of isopropanol and 3 μL GlycoBlue. Samples were centrifuged for 20 min at 21,000×g and supernatant removed. Pellets were washed using 80% ethanol, air dried for ˜30 min and resuspended in 10 mM Tris (pH 8) prior to storage at −80° C.

qPCR. Community growth was estimated by quantifying 16S rRNA gene copies from co-culture DNA. Reactions were prepared in triplicate using iTaq Universal SYBR Green Supermix (Bio-Rad) and standard 16S rDNA qPCR primers 338F and 518R(18). Reactions each contained 10 μL 2×SYBR Green, 2 μL each of 3 μM primers, and 5 μL H₂O. DNA from each sample was diluted to 10 ng/μL and 1 μL was added to each reaction. Amplification was performed using a CFX96 PCR System (Bio-Rad) with the following cycling conditions: 95° C. for 5 min followed by 40 cycles of 95° C. for 5 s and 60° C. for 30 s. Quantification cycle values were calculated using instrument software (CFX Manager, v. 3.1). Standard curves with a range from 5×106 to 5×102 16S rRNA gene copies were prepared using serial dilutions of DNA from Fusobacterium nucleatum ATCC 25586. Community growth was expressed as a percentage of 16S rRNA gene copy number relative to untreated controls.

### REFERENCES

1. A. L. Smith, S. B. Fiel, N. Mayer-Hamblett, B. Ramsey, J. L. Burns, Susceptibility testing of Pseudomonas aeruginosa isolates and clinical response to parenteral antibiotic administration: lack of association in cystic fibrosis. Chest 123, 1495-1502 (2003).
2. M. N. Hurley, A. H. Ariff, C. Bertenshaw, J. Bhatt, A. R. Smyth, Results of antibiotic susceptibility testing do not influence clinical outcome in children with cystic fibrosis. J Cyst Fibros 11, 288-292 (2012).
3. E. S. Cowley, S. H. Kopf, A. LaRiviere, W. Ziebis, D. K. Newman, Pediatric cystic fibrosis sputum can be chemically dynamic, anoxic, and extremely reduced due to hydrogen sulfide formation. MBio 6, e00767 (2015).
4. C. R. Bodem, L. M. Lampton, D. P. Miller, E. F. Tarka, E. D. Everett, Endobronchial pH: Relevance of aminoglycoside activity in gram-negative bacillary pneumonia. Am Rev Respir Dis 127, 39-41 (1983).
5. K. N. Kragh et al., Polymorphonuclear leukocytes restrict growth of Pseudomonas aeruginosa in the lungs of cystic fibrosis patients. Infect Immun 82, 4477-4486 (2014).
6. S. Tavernier et al., Community Composition Determines Activity of Antibiotics against Multispecies Biofilms. Antimicrob Agents Chemother 61 (2017).
7. L. Radlinski et al., Pseudomonas aeruginosa exoproducts determine antibiotic efficacy against Staphylococcus aureus. PLoS Biol 15, e2003981 (2017).
8. L. J. Sherrard et al., Production of extended-spectrum β-lactamases and the potential indirect pathogenic role of Prevotella isolates from the cystic fibrosis respiratory microbiota. Int J Antimicrob Agents 47, 140-145 (2016).
9. E. M. Adamowicz, J. Flynn, R. C. Hunter, W. R. Harcombe, Cross-feeding modulates antibiotic tolerance in bacterial communities. ISME J12, 2723-2735 (2018).
10. J. M. Flynn, D. Niccum, J. M. Dunitz, R. C. Hunter, Evidence and role for bacterial mucin degradation in cystic fibrosis airway disease. PLoS Pathog 12, e1005846 (2016).
11. S. M. Moskowitz, J. M. Foster, J. Emerson, J. L. Burns, Clinically feasible biofilm susceptibility assay for isolates of Pseudomonas aeruginosa from patients with cystic fibrosis. J Clin Microbiol 42, 1915-1922 (2004).
12. L. J. Sherrard et al., Antibiotic resistance in Prevotella species isolated from patients with cystic fibrosis. J Antimicrob Chemother 68, 2369-2374 (2013).
13. J. L. Connell, E. T. Ritschdorff, M. Whiteley, J. B. Shear, 3D printing of microscopic bacterial communities. Proc Natl Acad Sci USA 110, 18380-18385 (2013).
14. A. Venkataraman, M. A. Rosenbaum, J. J. Werner, S. C. Winans, L. T. Angenent, Metabolite transfer with the fermentation product 2,3-butanediol enhances virulence by Pseudomonas aeruginosa. ISME J 8, 1210-1220 (2014).
15. A. Korgaonkar, U. Trivedi, K. P. Rumbaugh, M. Whiteley, Community surveillance enhances Pseudomonas aeruginosa virulence during polymicrobial infection. Proc Natl Acad Sci USA 110, 1059-1064 (2013).
16. K. Duan, C. Dammel, J. Stein, H. Rabin, M. G. Surette, Modulation of Pseudomonas aeruginosa gene expression by host microflora through interspecies communication. Mol Microbiol 50, 1477-1491 (2003).
17. R. A. Whiley, E. V. Fleming, R. Makhija, R. D. Waite, Environment and colonisation sequence are key parameters driving cooperation and competition between Pseudomonas aeruginosa cystic fibrosis strains and oral commensal streptococci. PLoS One 10, e0115513 (2015).
18. D. Lane, “16S/23S rRNA sequencing” in Nucleic acid techniques in bacterial systematics, E. Stackebrant, M. Goodfellow, Eds. (John Wiley & Sons New York, 1991), pp. 115-175.

The present invention has been described in terms of one or more preferred embodiments, and it should be appreciated that many equivalents, alternatives, variations, and modifications, aside from those expressly stated, are possible and within the scope of the invention.

We claim:

1. A method for identifying an antibiotic suitable for treating polymicrobial infection, the method comprising:
(a) combining in one or more containers a biological sample from a patient with a polymicrobial infection, one or more antibiotics and a media with a nutrient source, wherein the biological sample comprises at least a first microorganism and a second microorganism, wherein at least the first microorganism is a pathogenic microorganism;
(b) incubating the one or more containers; and
(c) detecting the growth of the one or more microorganism in the one or more containers, wherein the reduction or inhibition of growth of at least one of the first or second microorganisms identifies the one or more antibiotics for treating the polymicrobial infection.

2. The method of claim 1, wherein the method further comprising:
(d) treating the patient with the polymicrobial infection with the one or more antibiotics identified in step (c).

3-4. (canceled)

5. The method of claim 1, wherein the second microorganism is an anaerobic, aerobic or mixed community of bacteria.

6. The method of claim 1, wherein detecting the growth of the microorganism comprises visual inspection for turbidity in the container, optical density measurement, colorimetric assay, optical imaging, quantitating a molecular, biochemical or metabolic marker of the bacteria, quantitating 16S RNA, or performing PCR.

7. (canceled)

8. The method of claim 1, wherein the first or second microorganism is a bacterial species from a genus selected from the group consisting of Pseudomonas, Staphylococcus, Haemophilus, Stenotrophomonas, Streptococcus, Mycobacterium, Enterococcus, Porphyromonas, Fusobacterium, Prevotella, Achromobacter, Veillonella, Rothia, Anaerococcus, Bacteroides, Corynebacterium, Cutibacterium, Finegoldia, Fusobacterium, Klebsiella, Lawsonella, Moraxella, Neisseria, Burkholderia cepacia complex, and Serratia.

9. The method of claim 1, wherein the patient from which the biological sample is collected has a disease or condition associated with or caused by a polymicrobial infection, wherein the disease or condition is selected from the group consisting of cystic fibrosis (CF), chronic obstructive pulmonary disorder (COPD), chronic sinusitis, gastrointestinal infection, chronic wound, septicemia, urinary tract infection, renal infection, an abscess, AIDS-related opportunistic infection, conjunctivitis, gastroenteritis, hepatitis, multiple sclerosis, otitis media, periodontal diseases, osteomyelitis, burn wound, urinary tract infection, vaginitis, medical device-related infection, pneumonia, keratitis, peritonitis, and diabetic foot wound infection.

10. (canceled)

11. The method of claim 1, wherein the media contains a single carbon source.

12. The method of claim 1, wherein the media comprises one or more nutrient sources selected from the group consisting of mucin, amino acids, a carbohydrate, a sugar, lipid, nucleic acids, and collagen.

13. The method of claim 1, wherein the media further comprises a gelling agent, wherein the gelling agent is a polymeric gelling agent, a cellulose-based gelling agent, a natural gelling agent, xanthan gum, guar gum, gellan, pectin, gelatin or agar.

14-15. (canceled)

16. The method of claim 13, wherein the natural gelling agent is agar.

17. (canceled)

18. The method of claim 1, wherein the one or more antibiotic is an aminoglycoside, a carbapenem, a cephalosporins, a fluoroquinolone, a glycopeptide, a lipoglycopeptide, a macrolide, a monobactam, an oxazolidinone, a penicillin, a polypeptide, a rifamycin, a chloramphenicol, a quinolones, a sulfonamide, a streptogramin, a tetracycline, a beta-lactam antibiotic, an ansamycin, a streptagramin, chloramphenicol, clindamycin, daptomycin, fosfomycin, lefamulin, metronidazole, mupirocin, nitrofurantoin, and tigecycline or a combination thereof.

19. The method of claim 1, wherein step (b) comprises incubating for a time sufficient to detect growth of the first microorganism and the second microorganism in the absence of a growth-inhibiting drug.

20. (canceled)

21. The method of claim 1, comprising adding the biological sample to two or more containers, wherein the containers comprise different concentrations of the antibiotic.

22. (canceled)

23. The method of claim 1, wherein the method comprises adding the biological sample to two or more containers, wherein each container comprises a different antibiotic.

24. The method of claim 1, further comprising homogenizing the biological sample prior to adding the sample to the one or more container.

25. The method of claim 1, further comprising
isolating a community of bacteria from a first portion of the sample to form a culture, wherein the community of microorganism comprise the second microorganism;
isolating one or more species of pathogenic microorganism from a second portion of the sample, wherein the one or more species of pathogenic microorganism comprise the first microorganism;
adding the community culture to the container comprising the media and antibiotic; and
adding the pathogenic bacteria to the container to perform the incubation step.

26. The method of claim 25, wherein the media further comprises a gelling agent, and the method further comprises incubating the combination to allow the gelling agent to thicken or solidify.

27. (canceled)

28. The method of claim 25, wherein the method further comprises determining the minimum inhibitory concentration (MIC) of the antibiotic required to inhibit growth of the community culture and determining the MIC of the antibiotic required to inhibit growth of the pathogenic bacteria.

29. A container or array of containers comprising (a) a homogenized biological sample obtained from a subject, wherein the biological sample is obtained from a fluid, tissue, or wound containing a polymicrobial infection comprising at least a first microorganism and a second microorganism, (b) one or more antibiotics, and (c) a single nutrient source that reflects the environment of the bacterial infection.

30-34. (canceled)

35. A high throughput method for determining a suitable antibiotic or small molecule for treatment of a polymicrobial infection in a subject, the method comprising
obtaining a biological sample from the subject;
adding a portion of the biological sample to each container in an array of containers, wherein each container in the array comprises an antibiotic or small molecule and a media containing a nutrient source;
incubating the containers; and
performing PCR to determine the growth of one or more bacteria in the container, thereby predicting the efficacy of the antibiotic or small molecule in treating the disease or condition.

36. (canceled)
